(19) United States
(12) Patent Application Publication
(10) Pub. No.: US 2009/0081245 A1
(43) Pub. Date: Mar. 26, 2009

(54) ASSAYS FOR DEVELOPMENT OF BORIS MUTANTS SUITABLE FOR VACCINE DEVELOPMENT AND SMALL MOLECULE INHIBITORS OF WILD-TYPE BORIS

(76) Inventors: Boris N. Reznik, Hollywood, FL (US); Christopher J. Dougherty, Hollywood, FL (US); Thomas Ichim, San Diego, CA (US)

Correspondence Address: The Law Office of Jane K. Babin;Professional Corporation, c/o Intellevate, P.O. Box 52050, Minneapolis, MN, 55402, US

(21) Appl. No.: 12/206,709

(22) Filed: Sep. 8, 2008

Related U.S. Application Data
(60) Provisional application No. 60/970,597, filed on Sep. 7, 2007.

(51) Int. Cl. A61K 39/00; C12Q 1/68; C12Q 1/02

(52) U.S. Cl. 424/184.1; 435/6; 435/29

(57) ABSTRACT

Disclosed are methods and compositions for developing mutants of the Brother of the Regulator of Imprinted Sites (BORIS) suitable for immunotherapeutic purposes. Said methods involve the mutagenesis and/or deletion of various sequences within wild-type BORIS protein with the objected aim of constructing nucleic acids and proteins encoded by said nucleic acids capable of eliciting immune responses while concurrently lacking oncogenicity. Methods of screening of small molecule inhibitors of wild-type BORIS activity are also provided.

### RELATED APPLICATIONS

The present application claims the benefit of priority to U.S. Provisional Application Number: 60/970,597 filed Sep. 17, 2007, the entire contents of which are incorporated herein by reference.

### FIELD OF THE INVENTION

The present invention relates to the field of therapeutic and pharmaceutical development, particularly the development of tumor vaccines. More particularly, the invention relates to methods for screening vaccine candidates in the field of tumor immunology. Even more particularly, the invention pertains to methods of identifying non-oncogenic mutants of a transcription factor suitable for generating therapeutic immune responses. The invention also relates to assay systems for screening molecules for the ability to inhibit an activity of wild-type BORIS.

### BACKGROUND OF THE INVENTION

The Brother of the Regulator of Imprinted Sites (BORIS) is an epigenetic acting transcription factor that derepresses expression of numerous tumor causative and tumor associated genes. It has been demonstrated that BORIS competes with a homologous protein termed CTCF for binding to sequences containing the CCCTC motif. In general terms, oncogenesis is reportedly associated with the displacement of the transcriptional silencer CTCF by BORIS which functions as a transcriptional activator. To date, expression of BORIS has been described in every cancer tissue evaluated, not only cell lines but also primary patient biopsy material. Given that the possibility of inducing immune responses to tumor associated transcription factors has already been demonstrated, it is of great interest to investigate the use of BORIS as a tumor antigen for vaccination.

Vaccination with BORIS would require the attenuation of its oncogenic properties, in a manner conceptually similar to the attenuation of pathogens for development of live vaccines. It has previously been reported that immunization with zinc finger-deleted BORIS molecules is sufficient to yield a cytotoxic T lymphocyte response, as well as production of cytokines associated with tumor regression such as interferon gamma. Mice vaccinated in this manner not only possessed proliferative recall responses to the wild-type BORIS protein but also were capable of mounting in vivo antitumor responses. In studies detailed in these reports utilized a mutated version of BORIS lacking all 11 zinc-fingers found on wild-type BORIS. To date, there is a paucity of data regarding the extent of wild-type BORIS protein that must to be modified or mutated to eliminate oncogenicity yet preserve immunogenicity toward the protein. The current invention provides methods of addressing this issue.

As a tumor antigen, BORIS is an attractive target for therapeutic intervention with conventional anti-tumor compounds, such as small molecule inhibitors. Accordingly it would be of interest to develop small molecule inhibitors of wild-type BORIS activity that could be used alone or in combination with anti-BORIS immunotherapy. Currently no methods of screening said small molecule inhibitors for suppression of BORIS oncogenic activity have been described.

### SUMMARY OF THE INVENTION

The present invention provides methods for identifying an immunotherapeutic candidate comprising the steps of: a) obtaining a polynucleotide encoding a wild-type BORIS polypeptide; b) generating a plurality of mutants of the polynucleotide of step a), wherein the polypeptides encoded by the mutant polynucleotides are deficient in DNA binding; c) identifying at least one DNA binding deficient mutant of step b) that is not oncogenic; d) immunizing an animal with the at least one non-oncogenic DNA binding deficient mutant of step c), thereby producing an immune response; e) screening the immune response generated in step d) for cross-reactivity with the wild-type BORIS polypeptide, wherein cross-reactivity to wild-type BORIS is identifies an immunotherapeutic candidate. The mutations leading to DNA binding deficiency may be localized in or proximal to a zinc finger encoding region of wild-type BORIS. The mutations may be generated by any method known in the art, such as by site-directed mutagenesis or PCT.

DNA binding of the mutants can be assessed by a variety of methods known in the art. For example, DNA affinity chromatography can be used, such as affinity chromatography to a DNA molecule containing a CCCTC motif. In certain embodiment, DNA binding can be assessed using an assay for transcription of at least a gene regulated by wild-type BORIS, such as hTERT; MAGE; GAGE; OCT-4; and c-myc. The ability to bind DNA can also be assessed indirectly by transfection into a non-transformed cell and measuring oncogenic transformation.

The identification of DNA binding deficient mutants that are not oncogenic, can be carried out by identifying a mutant that does not promote oncogenic transformation, as assessed by common hallmarks of oncogenic transformation such as growth factor independence, resistance to apoptosis, upregulation of matrix metalloprotease activity, or ability to form a tumor in an animal. In another embodiment of the invention, oncogenic transformation activity is assessed in a human cell, with the human cell being assessed for oncogenic transformation by transplantation into an immune deficient animal, such as a SCID mouse.

Immunizing the animal can be accomplished by administering DNA-binding deficient BORIS polypeptides in an immunogeneic (e.g., coadministration of an adjuvant) context to animals that are immune competent.

The immune response can, for example, be a T cell response, and can be measured e.g., by a proliferative recall response assay, a T cell cytokine recall response assay, or a T cell cytotoxic recall response assay. Recall responses can be assessed by restimulation of immune cells from an immunized animal with an antigen presenting cell that has been previously pulsed with the wild-type BORIS protein, nucleotides encoding said BORIS protein, or derivatives thereof. In certain embodiments, recall responses are assessed by restimulation of immune cells from an immunized animal with an antigen presenting cell that expresses the wild-type BORIS protein or a nucleotide encoding the wild-type BORIS protein. In yet further embodiments, recall responses are assessed by restimulation of immune cells from an immunized animal with a tumor cell that expresses the wild-type BORIS protein or a nucleotide encoding the wild-type BORIS protein.

Also provided by the present invention are methods for screening a molecule such as a small organic compound, for the ability to inhibit BORIS activity including the steps of contacting a BORIS reporter cell with the molecule, where the BORIS reporter cell expresses wild-type BORIS polypeptide and comprises a reporter construct containing a BORIS-responsive promoter operatively linked to a reporter polynucleotide, wherein expression of the reporter from the construct is indicative of BORIS activity; and measuring the level of expression of the reporter, where a reduction in the level of expression of the reporter as compared to a control, untreated reporter cell, indicates the ability to inhibit BORIS activity. In one embodiment, the compound is derived from a compound library.

The reporter cell can be, for example, a primary fibroblasts, primary lymphocytes, primary fibroblasts, HEK-293, or COS cell transformed with a wild-type BORIS expression vector and the marker is polynucleotide encodes green fluorescent protein, Firefly Luciferase, a luminescent protein, or a cell surface marker and expression of the marker can be measured, for example, by flow cytometry, fluorescent microscopy, magnetic separation, or immunoadhesion.

### DETAILED DESCRIPTION

It is to be understood that both the foregoing general description and the following detailed description are exemplary and explanatory only and are not restrictive of the invention, as claimed. It must be noted that, as used herein and in the appended claims, the singular forms include plural referents; the use of “or” means “and/or” unless stated otherwise. Thus, for example, reference to “a subject polypeptide” includes a plurality of such polypeptides and reference to “the agent” includes reference to one or more agents and equivalents thereof known to those skilled in the art, and so forth. Moreover, it must be understood that the invention is not limited to the particular embodiments described, as such may, of course, vary. Further, the terminology used to describe particular embodiments is not intended to be limiting, since the scope of the present invention will be limited only by its claims.

The section headings used herein are for organizational purposes only and are not to be construed as limiting the subject matter described. All documents, or portions of documents, cited in this application, including but not limited to patents, patent applications, articles, books, and treatises, are hereby expressly incorporated by reference in their entirety for any purpose.

Unless otherwise defined, all technical and scientific terms used herein have the same meaning as commonly understood by one of ordinary skill in the art to which this invention belongs. Suitable methods and materials are described below, however methods and materials similar or equivalent to those described herein can be used in the practice of the present invention. Thus, the materials, methods, and examples are illustrative only and not intended to be limiting. All publications, patent applications, patents, and other references mentioned herein are incorporated by reference in their entirety. In case of conflict, the present specification, including definitions, will control.

Standard techniques may be used for recombinant DNA, oligonucleotide synthesis, tissue culture and transfection (e.g., electroporation, lipofection, etc.). Enzymatic reactions and purification techniques may be performed according to manufacturer's specifications or as commonly accomplished in the art or as described herein. The foregoing techniques and procedures may be generally performed according to conventional methods well known in the art and as described in various general and more specific references that are cited and discussed throughout the present specification. See e.g., Sambrook et al. Molecular Cloning: A Laboratory Manual (2d ed., Cold Spring Harbor Laboratory Press, Cold Spring Harbor, N.Y. (1989)); Current Protocols in Molecular Biology (eds. Ausubel, et al., Greene Publ. Assoc., Wiley-Interscience, N.Y.); Harlow and Lane, Antibodies, A Laboratory Manual, Cold Spring Harbor Laboratory, Cold Spring Harbor, N.Y. (1988)) the entire contents of which are incorporated herein by reference for any purpose. Unless specific definitions are provided, the nomenclatures utilized in connection with, and the laboratory procedures and techniques of, analytical chemistry, synthetic organic chemistry, and medicinal and pharmaceutical chemistry described herein are those well known and commonly used in the art. Standard techniques may be used for chemical syntheses, chemical analyses, pharmaceutical preparation, formulation, and delivery, and treatment of patients.

While the present invention may be embodied in many different forms, specific illustrative embodiments are disclosed herein that exemplify the principles of the invention. It should be emphasized that the present invention is not limited to the specific embodiments illustrated.

The invention involves the introduction of specific changes in the DNA encoding the antigen to eliminate side effects and autoimmune reactions. In this context the following definitions apply.

“About” as used herein means that a number referred to as “about” comprises the recited number plus or minus 1-10% of that recited number. For example, “about” 100 nucleotides can mean 100 nucleotides, 99-101 nucleotides, or up to as broad a range as 90-100 nucleotides. Whenever it appears herein, a numerical range such as “1 to 100” refers to each integer in the given range; e.g., “1 to 100 nucleotides” means that the nucleic acid can contain only 1 nucleotide, 2 nucleotides, 3 nucleotides, etc., up to and including 100 nucleotides.

Wild-type “BORIS” or “the Brother of the Regulator of Imprinted Sites” protein, as used herein, refers to an epigenetically-acting zinc finger polypeptide present in mammalian testes and cancer cells, with an amino acid sequence that has greater than about 80% amino acid sequence identity, typically greater than 85% identity, often greater than 90% identity, and preferably 91%, 92%, 93%, 94%, 95%, 96%, 97%, 98% or 99% or greater amino acid sequence identity, to the BORIS amino acid sequence detailed in GenBank Accession No. AAM28645 (posted May 16, 2002), and is capable of transforming a mammalian cell.

The skilled artisan will be aware of methods for determining whether a polypeptide is capable of transforming a mammalian cell, such as by transfection of a nucleic acid encoding the variant into a cell and e.g. observing colony formation. Typically, cancer cells that express BORIS have the amino acid sequence of GenBank Accession No. AAM28645, or a variant containing one or more conservative amino acid substitutions, or a polymorphic variant thereof that is capable of transforming a mammalian cell.

Identity is determined over a region of at least 20, 50, 100, 200, 500, or more contiguous amino acids. The terms “identical” or percent “identity,” as used herein in the context of two or more nucleic acids or amino acid sequences, refer to two or more sequences or subsequences that are the same or have a specified percentage of amino acid residues or nucleotides that are the same when compared and aligned for maximum correspondence over a comparison window (i.e. region). The definition includes sequences that have deletions, insertions and substitutions and may also be applied to the complement of a sequence (e.g. “100% complementary” polynucleotides). Preferably, identity is measured over the length of the polynucleotide or polypeptide, but is typically measured over a region that is at least about 20 amino acids or nucleotides in length, and often over a region that is at least 50-100 amino acids or nucleotides in length.

To calculate percent sequence identity, two sequences are aligned and the number of identical matches of nucleotides or amino acid residues between the two sequences is determined. The number of identical matches is divided by the length of the aligned region (i.e., the number of aligned nucleotides or amino acid residues) and multiplied by 100 to arrive at a percent sequence identity value, which is usually rounded to the nearest integer. It will be appreciated that the length of the aligned region can be a portion of one or both sequences up to the full-length of the shortest sequence. It will be appreciated that a single sequence can align differently with other sequences and hence, can have different percent sequence identity values over each aligned region.

The alignment of two or more sequences to determine percent sequence identity can be performed manually, by visual alignment, or can use computer programs that are well known in the art. For example, the algorithm described by Altschul et al. (1997, Nucleic Acids Res., 25:3389 402) can be used. This algorithm is incorporated into BLAST (basic local alignment search tool) programs, available at ncbi.nlm.nih.gov on the World Wide Web. BLAST searches can be performed to determine percent sequence identity between a nucleic acid molecule or polypeptide of the invention and any other sequence or portion thereof.

Wild-type “BORIS gene” or “BORIS polynucleotide” refer to a polynucleotide sequence encoding a wild-type BORIS polypeptide, which is transcribed into an mRNA with at least about 80% nucleotide sequence identity, typically greater than 85% identity, often greater than 90% identity, and preferably 91%, 92%, 93%, 94%, 95%, 96%, 97%, 98% or 99% or greater nucleotide sequence identity to the BORIS cDNA sequence of GenBank Accession No. AF336042 (posted May 16, 2002).

“CTCF” as used herein refers to CCCTC-binding factor, a paralog of BORIS that is expressed in normal mammalian cells, and which typically has about 66% amino acid sequence identity to BORIS. “CTCF gene” refers to a polynucleotide sequence encoding a CTCF polypeptide, which is transcribed into an mRNA have a nucleotide sequence that has at least at least about 80% nucleotide sequence identity, typically greater than 85% identity, often greater than 90% identity, and preferably 91%, 92%, 93%, 94%, 95%, 96%, 97%, 98% or 99% or greater nucleotide sequence identity to the CTCF cDNA sequence of GenBank Accession No.:NM_{—}006565 (posted Jul. 20, 2008).

The terms “polynucleotide,” “nucleic acid,” and “nucleic acid molecule,” are used interchangeably herein to refer to polymeric forms of nucleotides of any length. The polynucleotides can contain deoxyribonucleotides, ribonucleotides, and/or their analogs. Polynucleotides can have any three-dimensional structure, and can perform any function, known or unknown. The term polynucleotide includes single-stranded, double-stranded, and triple helical molecules, and encompasses nucleic acids containing nucleotide analogs or modified backbone residues or linkages, which can be synthetic, naturally occurring, or non-naturally occurring, and which have similar binding properties as the reference nucleic acid. In particular, interfering RNAs (e.g., siRNA, shRNA) of the invention, can contain modifications or may incorporate analogs provided these do not interfere with the ability of the interfering RNA to inactivate homologous MRNA. Examples include replacement of one or more phosphodiester bonds with phosphorothioate linkages; modifications at the 2′-position of the pentose sugar in RNA, such as incorporation of 2′-O-methyl ribonucleotides, 2′-H ribonucleotides, 2′-deoxy-2′-fluoro ribonucleotides (e.g. 2′-deoxy-2′-fluorouridine), or 2′-deoxy ribonucleotides; incorporation of universal base nucleotides, 5-C-methyl nucleotides, inverted deoxyabasic residues, or locked nucleic acid (LNA), which contains a methylene linkage between the 2′ and the 4′ position of the ribose.

Exemplary embodiments of polynucleotides include, without limitation, genes, gene fragments, exons, introns, mRNA, tRNA, rRNA, interfering RNA, siRNA, shRNA, miRNA, anti-sense RNA, ribozymes, cDNA, recombinant polynucleotides, branched polynucleotides, plasmids, vectors, isolated DNA of any sequence, isolated RNA of any sequence, nucleic acid probes and primers.

“Oligonucleotide” refers generally to polynucleotides that are between 5 and about 100 nucleotides of single- or double-stranded DNA. For the purposes of this disclosure, the lower limit of the size of an oligonucleotide is two, and there is no upper limit to the length of an oligonucleotide. Oligonucleotides are also known as “oligomers” or “oligos” and can be prepared by any method known in the art including isolation from naturally-occurring polynucleotides, enzymatic synthesis and chemical synthesis.

The term “polypeptide,” refers to a polymer of amino acid residues of any length. Polypeptides can have any three-dimensional structure, and can perform any function, known or unknown. The term applies to amino acid polymers in which one or more amino acid residue is an artificial chemical mimetic of a corresponding naturally occurring amino acid, as well as to naturally occurring amino acid polymers and non-naturally occurring amino acid polymers. “Peptide” as used herein, refers to a polymer of amino acid residues that is less than 250, frequently less than 100 and often less than 50 amino acid residues in length. “Protein” as used herein, refers to peptide and polypeptide forms that may contain multiple amino acid polymer molecules and in particular to mature forms that may include tertiary and quaternary structures, post-synthetic modifications, coordinated metals, and the like.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid mimetics refers to chemical compounds that have a structure that is different from the general chemical structure of an amino acid, but that functions in a manner similar to a naturally occurring amino acid.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

The terms “conservatively modified variants” or “conservative variants” applies to both amino acid and nucleic acid sequences. With respect to particular nucleic acid sequences, conservatively modified variants refers to those nucleic acids which encode identical or substantially identical amino acid sequences; or for nucleic acids that do not encode an amino acid sequence, to nucleic acids that are substantially identical. As used herein, “substantially identical” means that two amino acid or polynucleotide sequences differ at no more than 10% of the amino acid or nucleotide positions, typically at no more than 5%, often at more than 2%, and most frequently at no more than 1% of the of the amino acid or nucleotide positions.

Because of the degeneracy of the genetic code, a large number of functionally identical nucleic acids encode any given protein. For instance, the codons GCA, GCC, GCG and GCU all encode the amino acid alanine. Thus, at every position where an alanine is specified by a codon, the codon can be altered to any of the alternate alanine codons without altering the encoded polypeptide. Such nucleic acid variations are “silent variations,” which are one type of conservatively modified variants. Nucleic acid sequences encoding polypeptides described herein also encompass every possible silent variation of the nucleic acid. The skilled artisan will recognize that each amino acid codon in a nucleic acid (except AUG, which is ordinarily the only codon for methionine, and TGG, which is ordinarily the only codon for tryptophan) can be varied at one or more positions to code for the same amino acid. Accordingly, each silent variation of a nucleic acid that encodes a polypeptide is implicit in each described sequence with respect to the expression product.

“Complementarity” refers to the ability of a nucleic acid to form hydrogen bond(s) with another polynucleotide sequence by either traditional Watson-Crick or other non-traditional types of base pairing. In reference to the nucleic molecules of the present invention, the binding free energy for a nucleic acid molecule with its target or complementary sequence is sufficient to allow the relevant function of the nucleic acid to proceed, e.g., enzymatic nucleic acid cleavage, RNA interference, antisense or triple helix inhibition. Determination of binding free energies for nucleic acid molecules is well known in the art. “Percent complementarity” refers to the percentage of contiguous residues in a nucleic acid molecule that can form hydrogen bonds (e.g., Watson-Crick base pairing) with another nucleic acid molecule. “Perfectly complementary” or “100% complementarity” means that all the contiguous nucleotides of a nucleic acid molecule will hydrogen bond with the same number of contiguous residues in a second nucleic acid molecule. “Substantial complementarity” and “substantially complementary” as used herein indicate that two nucleic acids are at least 90% complementary, typically at least 95% complementary, often at least 98% complementary, and most frequently at least 99% complementary over a region of more than about 15 nucleotides and more often more than about 19 nucleotides.

Regarding amino acid sequences, one of skill in the art will recognize that individual substitutions, deletions or insertions to a nucleic acid, peptide, polypeptide, or protein sequence which alters, inserts or deletes a single amino acid or a small percentage of amino acids in the encoded sequence is a “conservatively modified variant” where the alteration results in the substitution of an amino acid with a chemically similar amino acid. Conservative substitution tables detailing functionally similar amino acids are well known in the art. Such conservatively modified variants are in addition to and do not exclude functionally equivalent polymorphic variants, homologs, and alleles of the invention.

The following eight groups each contain amino acids that are conservative substitutions for one another: 1) Alanine (A), Glycine (G); 2) Aspartic acid (D), Glutamic acid (E); 3) Asparagine (N), Glutamine (Q); 4) Arginine (R), Lysine (K); 5) Isoleucine (I), Leucine (L), Methionine (M), Valine (V); 6) Phenylalanine (F), Tyrosine (Y), Tryptophan (W); 7) Serine (S), Threonine (T); and 8) Cysteine (C), Methionine (M) (see, e.g., Creighton, Proteins (1984)).

“Mutant” or “mutation” as used herein refers to a naturally occurring, or more particularly an intentional change in the nucleotide or amino acid sequence of a polynucleotide or polypeptide, respectively.

A “nonfunctional mutant” refers to a polypeptide that lacks any function of the wild-type polypeptide. “Lack of function” is intended to mean failing to perform any one of the activities that the wild-type molecule performs. Thus, for example, a BORIS mutant that is deficient in DNA binding (i.e. a DNA binding deficient mutant), re-establishment of paternal DNA-methylation pattern or oncogenic transformation is a non-functional mutant or lacks function. Non-oncogenic mutants of BORIS are also encompassed by the term nonfunctional mutant.

“Nonfunctional mutations” can be amino acid substitutions, deletions or additions in areas of the molecule involved in catalytic and/or binding interactions, or additions or deletions of nucleotides that cause frame shifts, thus destroying the required three dimensional structure. Mutations can be produced using common molecular techniques such as PCR, use of oligonucleotides, etc. (for example see Sambrook, Maniatis and Fritsch). Naturally occurring mutations can also be isolated from cell populations (for example see Sambrook, Maniatis and Fritsch).

A “nonfunctional mutant BORIS polypeptide” may have at least about 50%, 60% or 70% sequence identity with the wild-type BORIS polypeptide.

A “polynucleotide encoding a nonfunctional mutant form of BORIS” can be a polynucleotide having at least 50%, 60% or 70% sequence identity to wild-type BORIS

“Expression” or “gene expression” as used herein refers to the conversion of the information from a gene into a gene product. A gene product can be the direct transcriptional product of a gene (e.g., mRNA, tRNA, rRNA, antisense RNA, ribozyme, structural RNA, or any other type of RNA) or a protein produced by translation. According to the methods of the present invention, gene expression is typically measured by determining the amount of a polypeptide in the cell, such as by enzyme linked immunosorbent assay (ELISA), Western blotting, radioimmunoassay (RIA), immunefluorescence, fluorescence activated cell analysis (FACS) or other methods that utilize antibodies. Gene expression may also be detected using biochemical techniques for analyzing RNA such as Northern blotting, nuclease protection assays, reverse transcription, microarray hybridization, and the like, which are well known in the art. In other aspects of the invention, gene expression is determined by measuring an activity or property of the expressed gene product, such as an enzymatic activity, fluorescence or luminescence. , such as BORIS methylation activity, DNA binding activity, or cell transformation activity.

“Antibody” or “antibodies”, as used herein, include naturally occurring species such as polyclonal and monoclonal antibodies as well as any antigen-binding portion, fragment or subunit of a naturally occurring molecule, such as for example Fab, Fab′, and F(ab)₂ fragments of an antibody. Also contemplated for use in the methods of the invention are recombinant, truncated, single chain, chimeric, and hybrid antibodies, including, but not limited to, humanized and primatized antibodies, and other non-naturally occurring antibody forms.

A “zinc finger domain” refers to a small independently folded polypeptide domain that is structurally stabilized by coordination of one or more zinc ions. Fingers bind to three base pair subsites and specific contacts are mediated by amino acids in positions-1, 2, 3 and 6 relative to the start of the alpha helix.

The terms “tumor,” “cancer,” “neoplasm,” “neoplasia” and their etymological relatives are used interchangeably in the context of this application to refer generally to dysproliferative diseases and the attendant affected cells or cell masses. Dysproliferative cells referred to herein may express an immune-privileged antigen.

“Cancer” for example, as used herein, refers to any abnormal cell or tissue growth, e.g., a tumor, which can be malignant or non-malignant. Cancer is characterized by uncontrolled proliferation of cells that may or may not invade the surrounding tissue and, hence, may or may not metastasize to new body sites. Cancer encompasses carcinomas, which are cancers of epithelial cells (e.g. squamous cell carcinoma, adenocarcinoma, melanomas, and hepatomas). Cancer also encompasses sarcomas, which are tumors of mesenchymal origin, (e.g. osteogenic sarcomas, leukemias, and lymphomas). Cancers can involve one or more neoplastic cell type.

A “pharmaceutical composition” or “pharmaceutically acceptable composition” of modulators, polypeptides, or polynucleotides herein refers to a composition that usually contains a pharmaceutically acceptable carrier or excipient that is conventional in the art and which is suitable for administration into a subject for therapeutic, diagnostic, or prophylactic purposes. For example, compositions for oral administration can form solutions, suspensions, tablets, pills, capsules, sustained release formulations, oral rinses, or powders.

“Operably linked,” as used herein, means without limitation, that the RNA coding region of a construct is in the correct location and orientation with respect to a promoter such that expression of the gene will be effected when the promoter is contacted with the appropriate polymerase and any required transcription factors.

The present invention is based on the observation that tumor antigens may be efficacious targets for the development of cancer immunotherapeutics, such as vaccines. However, the side effect of oncogenic transformation by tumor antigens must be overcome in order to useful in such a capacity. The invention is also based on the observation that DNA binding is a requirement for oncogenic transformation by BORIS. Thus, the present invention provides methods for identifying non-oncogenic BORIS mutants that are deficient in DNA binding as a step in identifying immunotherapeutic candidates.

Cytotoxic T lymphocytes (CTLs) are effector T cells, usually CD8⁺ that can mediate the lysis of target cells bearing antigenic peptides associated with a MHC molecule. Other cytotoxic cells include gamma/delta and CD4⁺ NK 1.1⁺ cells. “Immune privilege” and “immune-privileged antigen” refer to the isolation of certain sites and antigens within the body from recognition and attach by the immune system. Thus an immune response is not normally generated to immune privileged antigens. Immune-privileged antigens expressed ectopically (i.e., outside of their normally immune-privileged sites) may result in autoimmunity or tumor immunity. Immune-privileged antigens are expressed by some tumors resulting in an immune response to both the tumor and to non-tumor sites expressing the same immune-privileged antigens.

“Antigen presenting cell” or “APC” as used herein, refers to cells including dendritic cells, macrophages, and B cells, that process and present antigenic peptides in association with class I or class II MHC molecules and deliver a co-stimulatory signal necessary for T cell activation.

A nonfunctional mutated BORIS molecule may be recognized as a non-self antigen expressed only in transformed tumor cells and can be used as an immunogen in a therapeutic context. The mutant form of BORIS may thus provide an ideal non-toxic immunotherapeutic, e.g., vaccine, because it should not have any undesirable side effects caused by its DNA-binding activity and/or native function. In other words, a mutant BORIS used for vaccination would have no functional activity and is present only as an immunogen (antigen). Unlike other tumor-specific antigens, BORIS is not expressed in the normal tissues in women. Furthermore, even though BORIS is expressed during the pubertal development of the normal testis in men, introduction and/or expression of a nonfunctional mutant BORIS should not be harmful, because the testis is an immune-privileged tissue (inaccessible for immune cells). In other words, the anti-BORIS immune response generated after immunization is not dangerous for normal cells and a BORIS vaccine does not induce autoimmunity. In addition, generation of a potent immune response is guaranteed because BORIS, unlike other tumor-specific antigens, is recognized as a foreign antigen. BORIS specific T cells are not deleted in thymus and recognize mutant BORIS as a non-self antigen and generate an immune response.

The present invention thus provides methods and compositions for identifying mutants of wild-type BORIS protein that are immunogenic but lack oncogenicity. The invention specifically provides methods for mutating the BORIS protein, assessing DNA binding, and testing immunogenicity of sequences that lack DNA binding activity.

In one aspect of the invention the present invention provides mutated BORIS polynucleotides that are defective in at least one of the eleven (11) interior Zinc Finger (ZF) domains of wild-type BORIS achieved, as may be generated by deletion, substitution or re-arrangement. The 11 ZFs are responsible for binding to the conserved CCCTC nucleotide sequence in the promoters of CTCF and BORIS regulated genes. Zinc fingers may be deleted from BORIS by PCR or re-arrangement of zinc fingers may be achieved through site-directed cloning. The skilled artisan will be aware of additional methods for producing mutations in polynucleotides encoding wild-type BORIS.

Generation of a BORIS-regulated reporter plasmid is provided in one embodiment of the invention to assay the efficacy of the BORIS mutants in an in-vitro assay using systems such as the Dual Luciferase Assay (DLA) System from Promega. An initial requirement for practice of this aspect of the invention is the cloning of promoters from BORIS regulated genes that contain the CCCTC region. Said CCCTC containing genes may include c-myc, INK4a, hTERT, Rasgfr, IGFII, and H19, p53, p21, Oct-4, NY-ESO-1 and MAGE-3 (Vatolin et al. Conditional expression of the CTCF-paralogous transcriptionalfactor BORIS in normal cells results in demethylation and derepression of MAGE-Al and reactivation of other cancer-testis genes. Cancer Res. 2005 Sep 1;65(17):7751-62) or may be genes associated with oncogenesis. The promoters for these genes are subsequently cloned into a vector which is operably linked to a marker protein that can be used for detection. In one specific embodiment, the CCCTC-containing promoter is cloned into the pGL3-Basic vector (Promega) which encodes Firefly Luciferase™ as the reporter gene for in-vitro analysis.

In one embodiment of the invention, DNA binding is characterized as an ability to activate expression from promoters containing the CCCTC-domain. To detect said DNA-binding, triple transfection of non-BORIS expressing cells may be performed using: 1. A plasmid encoding a mutant BORIS molecule being evaluated; 2. A plasmid encoding the CCCTC driven promoter operably controlling a detectable signal, such as the pGL3-CCCTC which encodes Firefly Luciferase; and 3. A plasmid encoding the TK-Renilla, which serves as a transfection control to normalize the DLA data. In positive controls, the plasmid encoding wild-type BORIS binds to and activates the transcription of Firefly Luciferase representing the 100% activity of all wild-type BORIS that contains 11 functional zinc fingers. In contrast, BORIS mutants with deleted zinc finger domains do not turn on, or partially activate said Firefly Luciferase expression by turning on the CCCTC-containing promoter. Said BORIS mutants lacking ability to turn on Luciferase Firefly expression are chosen as non-oncogenic variants.

In another embodiment, the methods of the invention developed for screening mutant BORIS oncogenic activity may also be exploited to assay for molecules that inhibit BORIS activity. Such molecules may include, for example, small organic compounds from a library or other collection. According to these methods of the invention, a BORIS reporter cell (that expresses wild-type BORIS polypeptide and contains a reporter construct containing a BORIS-responsive promoter operatively linked to a reporter polynucleotide) is contacted with test molecule, and the reporter activity is measured. A reduction in the level of expression of the reporter in the presence of the test molecule as compared a control that is not contacted with the test molecule, is an indication that the molecule inhibits BORIS activity.

In other embodiments, transfection of non-cancerous cells with BORIS mutants is performed in order to assess endowment of activities associated with oncogenesis. Said activities include: growth factor independence, telomerase upregulation, ability to divide past the Hayflick limit, apoptosis resistance, expression of metastasis-associated proteins, expression of matrix metalloproteases, and ability to form tumors in an animal.

In another embodiment of the invention, BORIS mutants selected based on lack of oncogenic activity are used to immunize animals to assess their ability to induce an immune response to wild-type BORIS. The immunization may be performed utilizing methods known in the art including administration of nucleic acids encoding BORIS mutants, administration of BORIS mutant proteins in the presence of an adjuvant, administration of pre-pulsed dendritic cells containing BORIS mutant, and administration of BORIS mutant together with a molecular adjuvant. Subsequent to immunization, techniques known in the art for assessment of immunogenicity are utilized. Examples of proliferative, cytokine, and cytotoxic recall responses are provided in the following two publications and incorporated by reference ((Ghochikyan et al. Elicitation of T cell responses to histologically unrelated tumors by immunization with the novel cancer-testis antigen, brother of the regulator of imprinted sites. J Immunol. 2007 Jan 1; 178(1):566-73. Loukinov D. Antitumor efficacy of DNA vaccination to the epigenetically acting tumor promoting transcription factor BORIS and CD80 molecular adjuvant. J Cell Biochem. 2006 Aug 1;98(5):1037-43)). Identification of BORIS mutants that selectively do not induce oncogenesis but are associated with stimulation of immunity towards wild-type BORIS are selected for further development. Exemplary steps in the further development of anti-BORIS immunotherapeutics are described in U.S. Patent Application Publication Nos. 2006/0286115 and 2003/0176378, the contents of which are incorporated by reference herein in their entirety for any purpose.

In one embodiment cDNA encoding wild-type BORIS is generated by RT-PCR from mRNA isolated from testis tissue. All or part of the DNA binding domain of the molecule is deleted, rearranged, or targeted for site-directed mutagenesis. The correct sequence is confirmed by automated nucleotide sequence analysis. The resulting molecule is non-functional for DNA binding activity.

The mutated cDNA is cloned into a pORF vector under control of the hEF1-HTLV promoter, however other expression vectors can be used. Here, the mutated cDNA is operably linked to a promoter and/or regulatory molecules that are capable of causing expression in the host cell. Viral vectors can be used including α-virus DNA or RNA vectors, adenoviruses and retroviruses (see Vasilevko, V. et al. (2003) Clin. Exp. Metastas. 20:489-98.; Leitner, W. W. et al. (2003) Nat Med 9:33-39; Ribas, A et al. (2002) Curr. Gene Ther 2:57-78).

In addition to the above, the invention encompasses using viral like particles encoding nonfunctional mutant BORIS molecules such as those from adenovirus, human hepatitis B, human hepatitis C, vaccinia virus, polyoviurs, etc. Recombinant viral proteins from different viruses have the useful property of self-assembling into virus-like particles (VLPs). These particles contain no viral nucleic acids and are therefore non-replicative, non-infectious and retain conformationally correct antigenic epitopes. VLP production has been shown in many experimental systems, such as mammalian cells, baculovirus-infected insect cells, yeasts, E. coli, cell free systems and transgenic plants. Importantly, vaccination with VLPs generates production of not only humoral but also cellular immune responses. VLPs infect professional APCs and subsequently induce protective cellular immune responses, including CD4⁺Th1 (type of CD4⁺T cells that helps CD8⁺T cells) and CD8⁺CTL responses. Thus, VLPs have clearly revealed an exceptional capacity to activate cellular immune responses (T cell responses). The potential use of VLPs as prophylactic vaccines is currently being assessed in a number of different clinical trials. Results from these trials have been encouraging with excellent tolerability and high immunogenicity reported in each trial. Generation of a VLPs vaccine composed of non-functional BORIS mutants may promote the induction of strong cellular immune responses against cancer cells expressing this tumor associated antigen. Hepatitis B virus (HBV) core antigen (HBcAg) and VSV are examples of suitable VLPs.

To generate a more robust cellular immune response, the mutated BORIS can be fused with molecular adjuvants such as B7 costimulatory molecules, beta-defensin ⅔, MIP3 α, IFNγ, cytokines, chemokines, etc. prior to cloning into the vector. Other suitable molecular adjuvants are include XCL1 (Lymphotactin α, SCM-1α, IL-1 α, IL-1β ATAC) XCL2 (Lymphotactin β, SCM-1 β, IL-2 ATAC) CCL1 (I-309, TCA3) IL-3 CCL2 (MCP-1, MCAF, JE) IL-4 CCL3 (MIP-1, αMIP-1 αS, IL-5 LD78 α) LD78 β (MIP-1 αP) IL-6 LD78γ IL-7 CCL4 (MIP-1 β) IL-9 CCL5 (RANTES) IL-10 CCL7 (MCP-3) IL-11 CCL8 (MCP-2) IL-12 CCL9 (MIP-1γ) IL13 CCL10 (CCF18) IL14 CCL11 (Eotaxin) IL-15 CCL12 (MCP-5) IL-16 CCL 13 (MCP-4, CK β 10) IL-17 CCL15 (HCC-2, Lkn-1, MIP-5, CC-2, IL-18 NCC-3, MIP-1.delta.) CCL16 (NCC-4, LEC, HCC-4, LMC, Mtn-1, IL-21 LCC-1, CK β 12) CCL17 (TARC) IL-23 CCL18 (DC-CK1, PARC, MIP-4, AMAC-1, TNF α CK β 7) CCL19 (exodus-3, ELC, MIP-3β, TNF β CK 11) CCL20 (exodus-1, MIP-3 α, LARC, IFN α ST38) CCL21 (exodus-2, SLC, 6-Ckine, TCA4, IFN β CK β 9) CCL22 (MDC, ABCD-1, DC/B-CK) IFNγ CCL23 (MIP-3, MPIF-1, CK β 8-1) M-CSF CCL24 (MPIF-2, CK β6, eotaxin-2) G-CSF CCL25 (TECK, Ck β 15) GM-CSF CCL26 (Eotaxin-3, MIP-4 α) MIF CCL27 (ALP, Skinkine, ILC, ESkine, CD46 (MCP) CTAK) CXCL8 (IL-8) CD27 (T14, S152) CXCL9 (mig) CD54 (ICAM-1) CXCL10 (γIP-10, crg-2) CD80 (B7-1, BB1) CXCL11 (H174, β-R1, I-TAC, IP-9) CD86 (B7-2, B70) CXCL12 (SDF-1 α, SDF-1 β, PBSF) CD134 (FLT3, STK-1) CXCL13 (BLC, BCA-1) CDw137 (4-1BB) CXCL14 (BRAK, bolekine) CDw150 (SLAM, IPO3) CX.sub.3CL1 (Fractalkine, neurotactin) CD153 (CD30L) Defensin (DF α, DF β) CD161 (NKR-PIA)

Alternatively, conventional adjuvants can be used such as Tween 80, 5% ethanol and Bupivacaine for DNA immunization. Other examples of conventional adjuvants include mineral salts (such as aluminium hydroxide and aluminium phosphate gels), oil emulsions and surfactant based formulations such as MF59, QS21, AS08 [SBAS2] (oil-in-water emulsion+MPL+QS21), Montanide ISA-51 and ISA-720, particulate adjuvants such as virosomes, AS04 ([SBAS4] A1 salt with MPL), ISCOMS, polylactide co-glycolide (PLG), microbial derivatives (natural and synthetic) including monophosphoryl lipid A (MPL), Detox (MPL+M.Phlei cell wall skeleton), AGP[RC-529], DC_Chol, OM-174 (lipid A derivative), CpG motifs, modified LT and CT (genetically modified bacterial toxins), endogenous immunomodulators such as GM-CSF, IL-12, Immudaptin, as well as all other chemokines, cytokines and costimulatory molecules listed in the table above and inert vehicles, such as gold particles.

Adjuvants can be either mixed with the polynucleotide encoding a nonfunctional mutant form of BORIS, a nonfunctional mutant BORIS polypeptide or a dendritic cell expressing a nonfunctional mutant BORIS polypeptide.

Additional polynucleotides can be included in the nonfunctional mutant BORIS constructs that encode peptide molecules known to enhance or promote presentation of the nonfunctional mutant BORIS by the professional antigen presenting cells (APC) cells of the MHC class pathway. One example of this is a construct the included coding sequence for the peptide transducing domain (PTD). In general, an immune response relies on native antigen processing and presentation. The tumor-associated antigens can be expressed in bacteria, yeast or mammalian cells, however protein antigens expressed in those systems likely will not maximally stimulate T cell responses (either CTL responses or Th1-biased responses) since the soluble exogenous proteins are processed mainly by the MHC class II pathway. In fact, many anti-tumor vaccines rely on the induction of CD8⁺ CTL, but this usually requires that the protein is synthesized within the cytosol of APC. Unfortunately, in general the plasma membranes of eukaryotic cells are impermeable to the majority of proteins. It has recently been shown, however, that foreign proteins fused with the protein-transducing domain (PTD) can penetrate the plasma membrane, allowing the proteins to accumulate within the cells. This enhances the presentation of foreign peptides by the MHC class I molecules of APCs to the antigen-specific CD8⁺ T cells.

### Vaccination/Immunization

Vaccine formulations of the present invention include a typically immunogenic amount of a polynucleotide encoding a nonfunctional mutant BORIS polypeptide, a nonfunctional mutant BORIS polypeptide or a dendritic cell expressing a nonfunctional mutant BORIS polypeptide in combination with a pharmaceutically acceptable carrier. Mimeotopes, which are polypeptides of unrelated sequence but with a 3-dimensional structure corresponding to the nonfunctional mutant BORIS polypeptide and that immunologically function in an identical manner can be used. Mimeotopes, which are any biological molecule that is unrelated to BORIS structure, but has identical 3-D antigenic epitope and can be recognized by anti-BORIS T cells.

An “immunogenic amount” is an amount of the polypeptide encoding a nonfunctional mutant BORIS polypeptide, nonfunctional mutant BORIS polypeptide or a dendritic cell expressing a nonfunctional mutant BORIS polypeptide sufficient to evoke an immune response in the subject to which the vaccine is administered. The amount administered is an amount that produces an immune response, preferably an immune response of a desired magnitude. Exemplary pharmaceutically acceptable carriers include, but are not limited to, sterile pyrogen-free water and sterile pyrogen-free physiological saline solution.

Immunotherapeutic, e.g. vaccine, formulations indentified by the methods of the present are contemplated to be suitable for patients diagnosed with at least one type of cancer including, but not limited to, breast, prostate, ovarian, gastric, liver, endometrial, glioma, colon, and/or esophageal cancer. The immunotherapeutic formulations of the present invention are also suitable for patients known to have a genetic susceptibility to cancer. In addition, the immunotherapeutic formulations identified by the methods of the present invention will be suitable for the general population at large, including those without cancer or without a genetic susceptibility to cancer, who wish to invoke protection against contracting at least one type of cancer that expresses the wild-type BORIS protein, polypeptide or peptide.

Administration of the vaccine formulation may be carried out by any suitable means, including parenteral injection (such as intraperitoneal, subcutaneous, or intramuscular injection), intradermal, intravenous, nasal, rectal, vaginal or to an airway surface. Topical application of the virus to an airway surface can be carried out by intranasal administration (e.g. by use of dropper, swab, or inhaler which deposits a pharmaceutical formulation intranasally). Topical application of the virus to an airway surface can also be carried out by inhalation administration, such as by creating respirable particles of a pharmaceutical formulation (including both solid particles and liquid particles) containing the replicon as an aerosol suspension, and then causing the subject to inhale the respirable particles. Methods and apparatus for administering respirable particles of pharmaceutical formulations are well known, and any conventional technique can be employed. An “immunogenic amount” is an amount of the replicon particles sufficient to evoke an immune response in the subject to which the vaccine is administered.

When RNA or DNA is used as an immunotherapeutic, e.g. vaccine, the RNA or DNA can be administered directly using techniques such as delivery on gold beads (gene gun), delivery by liposomes, or direct injection, among other methods known to people in the art. Any one or more constructs or replicating RNA can be use in any combination effective to elicit an immunogenic response in a subject. Generally, the nucleic acid vaccine administered may be in an amount that will induce a desired immune response, and the degree of protection desired. Precise amounts of the vaccine to be administered may depend on the judgment of the practitioner and may be peculiar to each subject and antigen.

The immunotherapeutic may be given in a single dose schedule, or preferably a multiple dose schedule in which a primary course of vaccination may be with 1-10 separate doses, followed by other doses given at subsequent time intervals required to maintain and or reinforce the immune response, for example, at 1-4 months for a second dose, and if needed, a subsequent dose(s) after several months. Examples of suitable immunization schedules include: (i) 0, 1 months and 6 months, (ii) 0, 7 days and 1 month, (iii) 0 and 1 month, (iv) 0 and 6 months, or other schedules sufficient to elicit the desired immune responses expected to confer protective immunity, or reduce disease symptoms, or reduce severity of disease.

What is claimed is:

1. A method for identifying an immunotherapeutic candidate comprising the steps of:
a) obtaining a polynucleotide encoding a wild-type BORIS polypeptide;
b) generating a plurality of mutants of the polynucleotide of step a), wherein the polypeptides encoded by the mutant polynucleotides are deficient in DNA binding;
c) identifying at least one DNA binding deficient mutant of step b) that is not oncogenic;
d) immunizing an animal with the at least one non-oncogenic DNA binding deficient mutant of step c), thereby producing an immune response;
e) screening the immune response generated in step d) for cross-reactivity with the wild-type BORIS polypeptide, wherein cross-reactivity to wild-type BORIS is identifies an immunotherapeutic candidate.

2. The method of claim 1, wherein at least one of the plurality of mutants generated in step b) is localized in or proximal to a zinc finger encoding region.

3. The method of claim 1, wherein generating the plurality of mutants mutations is performed by site-directed mutagenesis or PCT.

4. The method of claim 1, wherein the polypeptides encoded by the mutant polynucleotides are assessed for DNA binding by affinity chromatography to a DNA molecule containing a CCCTC motif.

5. The method of claim 1, wherein the polypeptides encoded by the mutant polynucleotides are assessed for DNA binding ability using an assay for transcription of at least one gene regulated by wild-type BORIS.

6. The method of claim 5, wherein the at least one gene regulated by wild-type BORIS is selected from hTERT; MAGE; GAGE; OCT-4; and c-myc.

7. The method of claim 1, wherein step c) comprises identifying at least one DNA binding deficient mutant that does not promote oncogenic transformation.

8. The method of claim 7, wherein oncogenic transformation comprises at least one of: growth factor independence, resistance to apoptosis, upregulation of matrix metalloprotease activity, ability to form a tumor in an animal.

9. The method of claim 1, wherein immunizing an animal comprises administering DNA-binding deficient polypeptides in an immunogeneic context to animals that are immune competent.

10. The method of claim 9, wherein the immunogeneic context comprises coadministration of an adjuvant.

11. The method of claim 1, wherein the immune response is a T cell response.

12. The method of claim 11, wherein the T cell response is measured by a proliferative recall response assay, a T cell cytokine recall response assay, or a T cell cytotoxic recall response assay.

13. The method of claim 12, wherein recall responses are assessed by restimulation of immune cells from an immunized animal with an antigen presenting cell that has been previously pulsed with the wild-type BORIS protein, nucleotides encoding said BORIS protein, or derivatives thereof.

14. The methods of claims 12, wherein recall responses are assessed by restimulation of immune cells from an immunized animal with an antigen presenting cell that expresses the wild-type BORIS protein or a nucleotide encoding the wild-type BORIS protein.

15. The methods of claim 12, wherein recall responses are assessed by restimulation of immune cells from an immunized animal with a tumor cell that expresses the wild-type BORIS protein or a nucleotide encoding the wild-type BORIS protein.

16. A method for screening a molecule for the ability to inhibit BORIS activity comprising the steps of:
a) contacting a BORIS reporter cell with the molecule, wherein the BORIS reporter cell expresses wild-type BORIS polypeptide and comprises a reporter construct containing a BORIS-responsive promoter operatively linked to a reporter polynucleotide, wherein expression of the reporter from the construct is indicative of BORIS activity;
b) measuring the level of expression of the reporter, wherein a reduction in the level of expression of the reporter as compared to a control, untreated reporter cell, indicates the ability to inhibit BORIS activity.

17. The method of claim 16, wherein the molecule is a small organic compound.

18. The method of claim 17, wherein the compound is derived from a compound library.

19. The method of claim 18, wherein the reporter cell is a primary fibroblasts, primary lymphocytes, primary fibroblasts, HEK-293, or COS cell transformed with a wild-type BORIS expression vector and the marker is polynucleotide encodes green fluorescent protein, Firefly Luciferase, a luminescent protein, or a cell surface marker.

20. The method of claim 19, wherein said expression of the marker is measured by flow cytometry, fluorescent microscopy, magnetic separation, or immunoadhesion.
